(19) United States
(12) Patent Application Publication
(10) Pub. No.: US 2017/0189671 A1
(43) Pub. Date: Jul. 6, 2017

(54) ELECTRICAL STIMULATION APPARATUS

(71) Applicants: Quanqin Dai, Diamond Bar, CA (US); Pu Jiang, Shenzhen (CN)

(72) Inventors: Quanqin Dai, Diamond Bar, CA (US); Pu Jiang, Shenzhen (CN)

(73) Assignee: JKH Health Co., Ltd., Shenzhen (CN)

(21) Appl. No.: 15/399,668

(22) Filed: Jan. 5, 2017

(30) Foreign Application Priority Data
Jan. 5, 2016 (CN) 201620007707.X

(51) Int. Cl. A61N 1/04; A61N 1/36

(52) U.S. Cl. CPC A61N 1/0484; A61N 1/36021; A61N 1/0452; A61N 1/36003

(57) ABSTRACT

An electrical stimulation apparatus comprises a control panel, and a stimulating accessory is electrically connected thereto. The control panel further has a power supply, a control unit and a wireless communication unit, and a shell covers the control panel. The electrical stimulation apparatus of the present invention is configured to be operated conveniently through a connected control terminal or directly through the control panel thereof, and once the electrical stimulation apparatus is connected to a control terminal, the parameters in both the apparatus and control terminal are configured to be synchronized to facilitate use and adjustment. The stimulating accessory is wearable, comfortable and convenient to use.

### FIELD OF THE INVENTION

The present invention relates to an electrical stimulation apparatus, and more particularly to an electrical stimulation apparatus capable of providing muscle stimulation and therapy through electric current.

### BACKGROUND OF THE INVENTION

Currently, researchers have found that the electric current can be applied to the medical and body-training fields, and there are two main kinds of electrical stimulation apparatuses, TENS (Transcutaneous Electrical Nerve Stimulation) and EMS (Electrical Muscle Stimulation), which are most commonly used for relieving pain and stimulating muscles of a user. TENS is usually used to provide a small and appropriate amount of electric current to relieve pains for a user while EMS is applied to use electric current to stimulate muscle strength and prevent muscle atrophy.

However, the conventional electrical stimulation apparatus can only provide one-way operation. It means that through connecting with a mobile or 3C (Computer/Communication/Consumer) device, the conventional electrical stimulation apparatus can be operated by a user through a remote or an APP (Application software) installed in the mobile or 3C device, but cannot be operated after disconnected from the mobile or 3C device. Thus, it may be inconvenient when user's mobile or 3C device is out of battery, and the situation limits the practicability of the apparatuses. Also, the conventional electrical stimulation apparatus is normally used along with a sticky gel electrode accessory that may need to be replaced frequently. Therefore, there remains a need for a new and improved design for an electrical stimulation apparatus to overcome the problems presented above.

### SUMMARY OF THE INVENTION

The present invention provides an electrical stimulation apparatus which comprises a control panel, and a stimulating accessory is electrically connected thereto. The control panel further has a power supply, a control unit and a wireless communication unit, and a shell covers the control panel. Wherein the control unit is configured to control the stimulating accessory, and the wireless communication unit is electrically connected to the control unit. Moreover, a power switch, an interaction portion and connectors are respectively installed with the shell. Wherein the power switch is configured to control the on/off operation of the power supply while the interaction portion electrically connected to the control unit is configured to respond to the operating signal sending from a user, and the connectors are configured to electrically connect to the stimulating accessory.

In one embodiment, the control unit comprises an IC chip which is preset to respond to operating signals from the interaction portion or a control terminal to control the stimulating accessory, or to send parameters according to received signals back to the connected control terminal, and wherein the control terminal is connected to the electrical stimulation apparatus.

In another embodiment, the wireless communication unit comprises a Bluetooth, Wi-Fi, radio frequency, or alternative module and is configured to have bi-directional communication with a preset control terminal, and the wireless module is electrically connected with the IC chip.

In still another embodiment, the control panel optionally comprises a display/indicator which is electrically connected to the control unit and configured to display control parameters of the control unit.

In a further embodiment, the optional display/indicator comprises at least a LED light which is configured to be activated corresponding to control parameters of the control unit.

In an advantageous embodiment, the stimulating accessory has a main portion which is configured to be attached or covers on a whole or a portion of a user's body, and the main portion has at least two electrodes which are insulated from each other. Wherein each of the electrodes has a connecting portion which is configured to electrically connect to the connector of the control panel.

In still a further embodiment, the electrodes are made of a conductive soft material (such as conductive fabric), and each of the electrodes has a conductive engaging portion formed at an outer side thereof, and a conductive engaging slot formed at the connector of the control panel is configured to engage with the engaging portion of the electrode.

In yet a further embodiment, the engaging portion of the electrode comprises a conductive fastener and a stub which are respectively formed at an outer surface and an inner surface of the electrode, and the engaging slot at the connector of the control panel has at least a conductive fastener which is configured to engage with the conductive fastener of the electrode on the stimulating accessory.

In a particular embodiment, the stimulating accessory is made of at least a conductive soft material (such as conductive fabric) and at least an insulated soft material (such as insulated fabric), which are sewed, knitted, or attached together.

In a preferred embodiment, the stimulating accessory is a conductive item which is wearable or configured to attach to a user's body such as a conductive sock, a conductive kneecap, a conductive piece of shorts, a conductive shirt, a conductive suit, a conductive glove, a conductive facial mask, a conductive shoe, a conductive arm/leg sleeve and a conductive waistband.

Comparing with conventional electrical stimulation apparatus, the present invention is advantageous because: (i) the electrical stimulation apparatus of the present invention is configured to be operated conveniently through a connected control terminal or directly through the control panel thereof; (ii) once the electrical stimulation apparatus is connected to a control terminal, the parameters in both the apparatus and control terminal are configured to be synchronized to facilitate use and adjustment; and (iii) the stimulating accessory is wearable, comfortable and convenient to use, compared to the conventional sticky gel electrode accessory that requires frequent replacements and spending.

### BRIEF DESCRIPTION OF THE DRAWINGS

FIG. 1 is a schematic view of an electrical stimulation apparatus in the present invention.

FIG. 2 is a three-dimensional exploded view of a stimulating accessory of the electrical stimulation apparatus of the present invention in the second embodiment.

FIG. 3 is a three-dimensional exploded view of a stimulating accessory of the electrical stimulation apparatus of the present invention in the third embodiment.

FIG. 4 is a three-dimensional exploded view of a stimulating accessory of the electrical stimulation apparatus of the present invention in the fourth embodiment.

FIG. 5 is a front view of a stimulating accessory of the electrical stimulation apparatus of the present invention in the fifth embodiment.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications that might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

In order to further understand the goal, characteristics and effect of the present invention, a number of embodiments along with the drawings are illustrated as following:

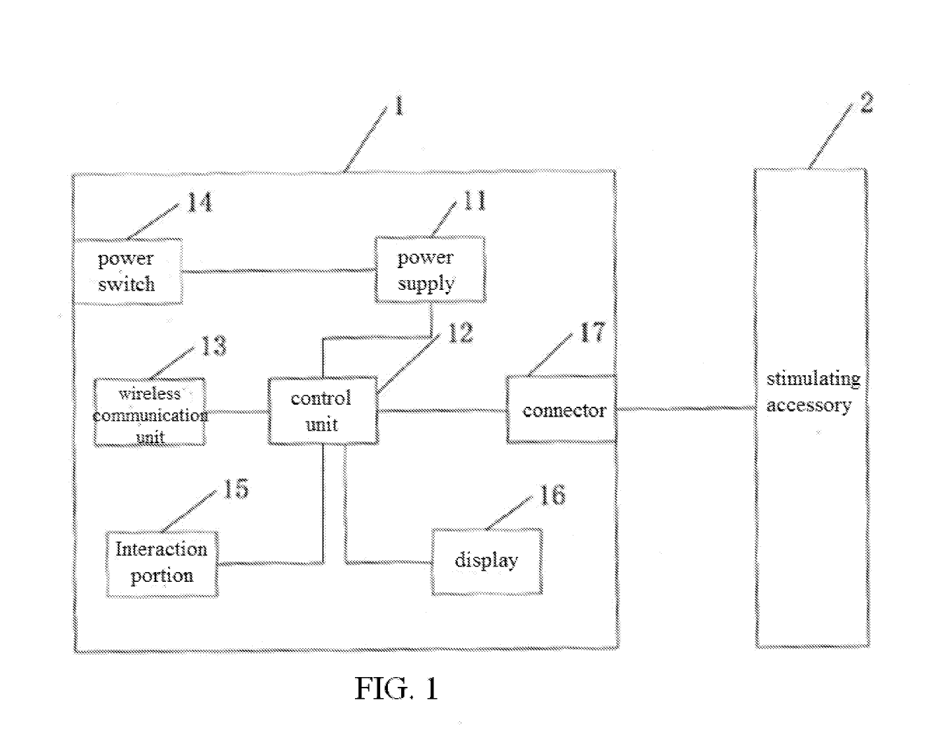

Referring to FIG. 1, the present invention provides an electrical stimulation apparatus which comprises a control panel (1), and a stimulating accessory (2) is electrically connected thereto. The control panel (1) further has a power supply (11), a control unit (12) and a wireless communication unit (13), and a shell covers the control panel (1). Wherein the control unit (12) is configured to control the stimulating accessory (2), and the wireless communication unit (13) is electrically connected to the control unit (12). Moreover, a power switch (14), an interaction portion (15) and a connector (17) are respectively installed with the shell. Wherein the power switch (14) is configured to control the on/off operation of the power supply (11) while the interaction portion (15) electrically connected to the control unit (12) is configured to respond to the operating signal sending from a user, and the connector (17) is configured to electrically connect to the stimulating accessory (2). In one embodiment, the control panel (1) optionally comprises a display/indicator (16) which is electrically connected to the control unit (12) and configured to display control parameters of the control unit (12).

In actual application, the stimulating accessory (2) is configured to be attached to and apply the electric current to a whole or a portion of a user's body according to preset parameters of the control unit (12). The control unit (12) is configured to provide different electric currents for different purposes such as relieving pains and/or stimulating the muscles of the user. Generally, the interaction portion (15) has a plurality of function buttons which are configured to allow a user to input and adjust parameters after turning on the control panel (1) through the power switch (14). For example, the interaction portion (15) may have a “+” button and a “−” button respectively for increasing and reducing the current intensity. The buttons on the interaction portion (15) are configured to create different signals to the control unit (12) for achieving different therapeutic effects for a user. Also, the optional display/indicator (16) is configured to display the use states of the control unit (12). Since the electrical stimulation apparatus of the present invention comprises the wireless communication unit (13), a user can connect his/her control terminal such as a mobile and a 3C device to the electrical stimulation apparatus, and control and realize different functions of the electrical stimulation apparatus through a remote or a corresponding APP (Application software) installed in the control terminal. As a result, the user can set and control operations of the electrical stimulation apparatus before or during the therapeutic and body-training processes.

In one embodiment, the electrical stimulation apparatus of the present invention is configured to be directly operated through the control panel (1) or be controlled through a remote or an APP installed in a control terminal when connected to the control terminal, thus improving the applicability thereof.

Wherein the control unit (12) comprises an IC chip which is preset to respond to operating signals from the interaction portion (15) or a control terminal to control the stimulating accessory (2), or to send parameters according to received signals back to the connected control terminal, and wherein the control terminal is connected to the electrical stimulation apparatus of the present invention.

In one embodiment, the IC chip of the electrical stimulation apparatus has a function of sending control parameters back to a control terminal. Specifically, when a control terminal is connected to the electrical stimulation apparatus of the present invention, the IC chip is configured to send its preset control parameters to the control terminal such that a user is able to set his/her preferred functions before or during the therapeutic and body-training processes. Thus, when the electrical stimulation apparatus of the present invention is connected to a control terminal, control parameters in a remote or an APP that is installed in the control terminal, and in the electrical stimulation apparatus are configured to be synchronized. As a result, the user can quickly choose and start his/her favorite therapeutic and body-training processes saved in the control terminal after the control terminal is connected to the electrical stimulation apparatus, which is convenient for use.

In another embodiment, the wireless communication unit (13) comprises a Bluetooth or alternative module and is configured to have bi-directional communication with a preset control terminal, and the Bluetooth module is electrically connected with the IC chip.

In still another embodiment, the optional display/indicator (16) comprises at least a LED light which is configured to be activated corresponding to control parameters of the control unit (12).

Based on any mentioned embodiments, the stimulating accessory (2) has a main portion which is configured to be attached or covers on a whole or a portion of a user's body, and the main portion has at least two electrodes which are insulated from each other. Wherein each of the electrodes has a connecting portion which is configured to electrically connect to the connector (17) of the control panel (1).

In one embodiment, the electrodes are made of a conductive soft material (such as conductive fabric), and each of the electrodes has a conductive engaging portion formed at an outer side thereof, and a conductive engaging slot formed at the connector (17) of the control panel (1) is configured to engage with the engaging portion of the electrode. In a preferable embodiment, the conductive engaging portion of the electrode comprises at least a conductive fastener and a stub which are respectively formed at an outer surface and an inner surface of the electrode, and the engaging slot at the connector of the control panel has at least a conductive fastener which is configured to engage with the conductive fastener of the electrode on the stimulating accessory (2). The stimulating accessory (2) is made of at least a conductive soft material (such as conductive fabric) and at least an insulated soft material (such as insulated fabric), which are sewed, knitted, or attached together.

In a preferable embodiment, the electrodes are made of conductive fabric which has soft texture and meets the conductive requirement. Thus, the single electrode is configured to be made easily and convenient to be worn. In another embodiment, the electrodes are made of conductive gel, silicone or other conductive materials.

In order to easily wear, preferably, the stimulating accessory (2) is made of at least a conductive soft material (such as conductive fabric) and at least an insulated soft material (such as insulated fabric) sewed, knitted, or attached together. Also, to ensure that the control panel (1) is electrically connected to the stimulating accessory (2), each of the electrodes made of a conductive soft material (such as conductive fabric) has a conductive fastener which is configured to engage with a conductive fastener secured on the control panel (1), thus allowing the control panel (1) to attach to the stimulating accessory (2). As a result, the control panel (1) is configured to give the output current to the electrodes to form a circuit between the user's body and the control panel (1), thereby achieving the therapeutic and body-training effects.

In one embodiment, the stimulating accessory (2) is a conductive item which is wearable or configured to attach to a user's body such as a conductive sock, a conductive kneecap, a conductive piece of shorts, a conductive shirt, a conductive suit, a conductive glove, a conductive facial mask, a conductive shoe, a conductive arm/leg sleeve and a conductive waistband.

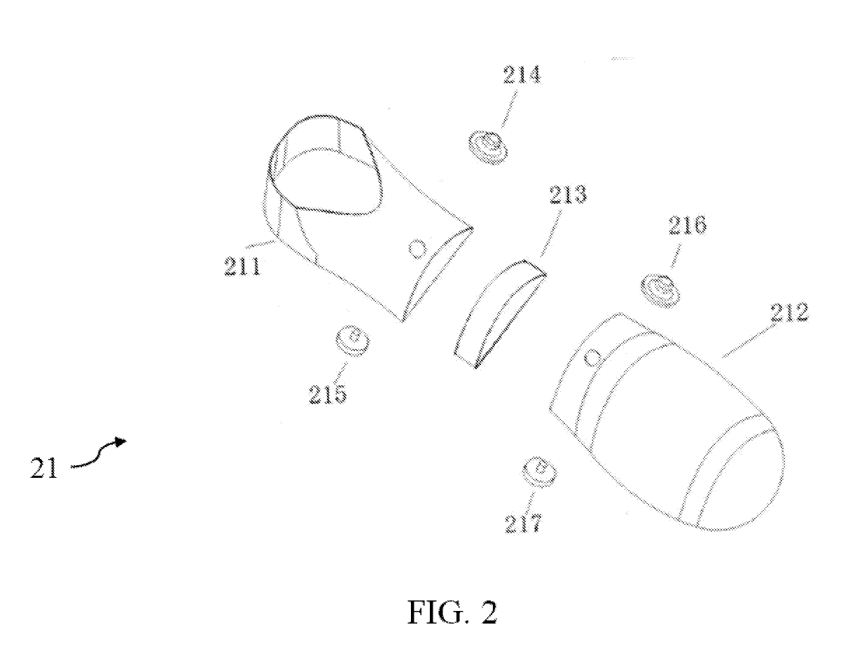

Referring to FIGS. 2 to 5, a conductive sock (21), a conductive kneecap (22), a conductive piece of shorts (23), and a conductive shirt (24) are respectively presented in the present invention as examples of embodiments of the stimulating accessory (2).

As shown in FIG. 2, in one embodiment, the conductive sock (21) comprises a first electrode (211) and a second electrode (212), which are made of conductive fabric to respectively be a rear portion and a front portion of the conductive sock. The first electrode (211) and the second electrode (212) are connected through an insulated fabric (213) to keep insulation therebetween. Wherein first electrode (211) further has a first metal male fastener (214) which is secured thereon through a first metal stub (215), and the second electrode (212) comprises a second metal male fastener (216) which is secured thereon through a second metal stub (217).

In the embodiment, the control panel (1) has at least one pair of separated metal female fasteners formed on a surface thereof, and a separated distance between the first metal male fastener (214) and the second metal male fastener (216) is arranged as same as a separated distance between the two corresponding female fasteners of the control panel (1).

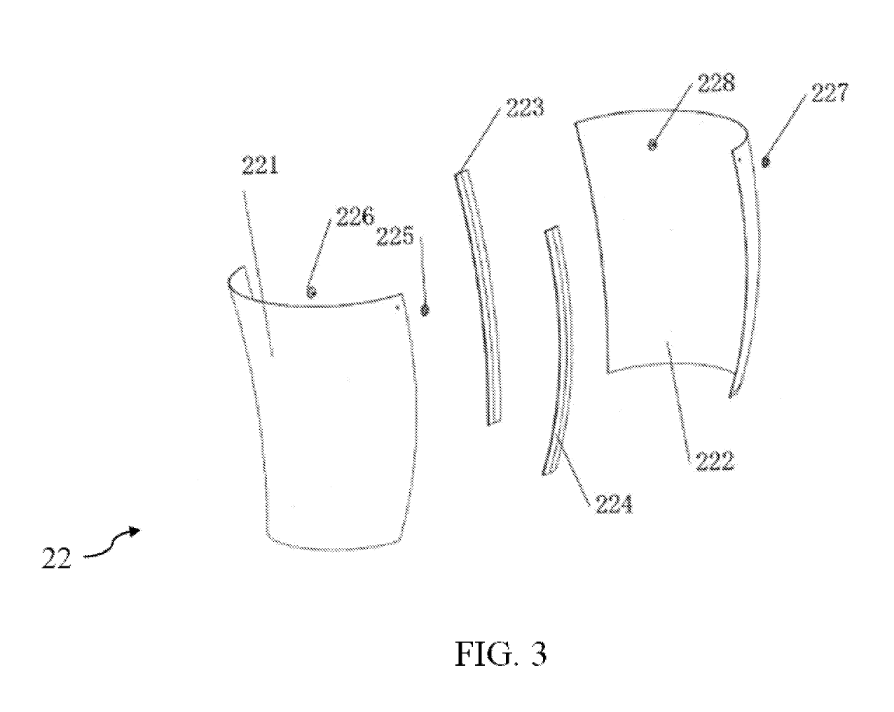

As shown in FIG. 3, in another embodiment, the conductive kneecap (22) comprises a first electrode (221) and a second electrode (222) which are made of conductive fabric and connected through a first insulated fabric (223) and a second insulated fabric (224) to keep insulation therebetween. Wherein the first electrode (221) has a first metal male fastener (225) which is secured thereon through a first metal stub (226) while the second electrode (222) comprises a second metal male fastener (227) which is secured thereon through a second metal stub (228).

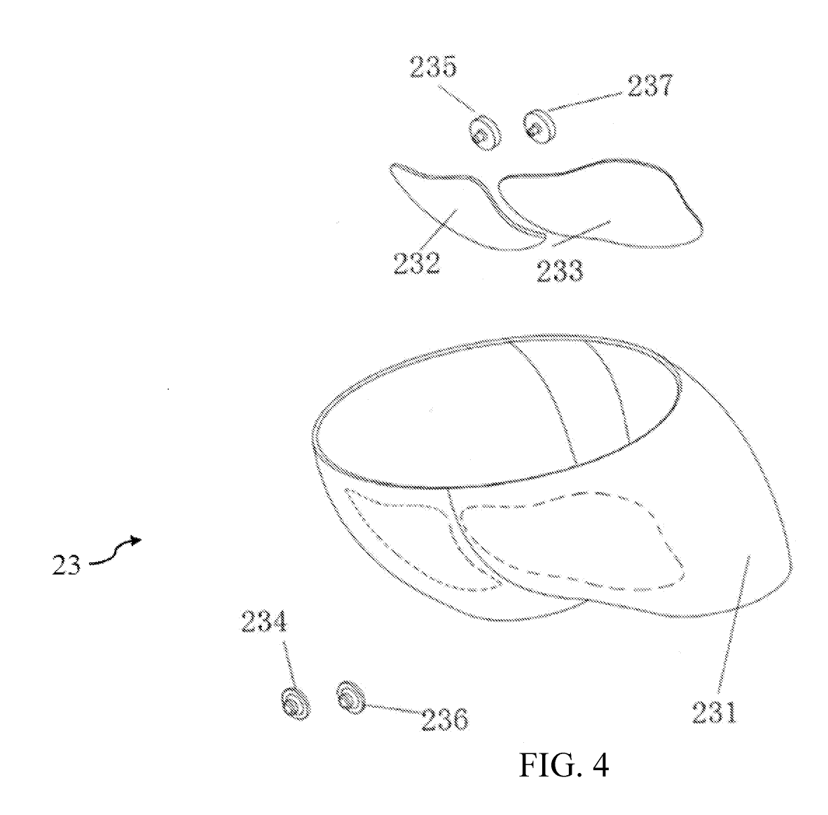

As shown in FIG. 4, in still another embodiment, the conductive shorts (23) comprise a main body (231) which is made of insulated fabric, and the main body (231) has a first electrode (232) and a second electrode (233) which are made of conductive fabric and respectively located at positions corresponding to user's hips. Wherein the first electrode (232) has a first metal male fastener (234) which is secured thereon through a first stub (235) while the second electrode (233) comprises a second metal male fastener (236) which is secured thereon through a second stub (237).

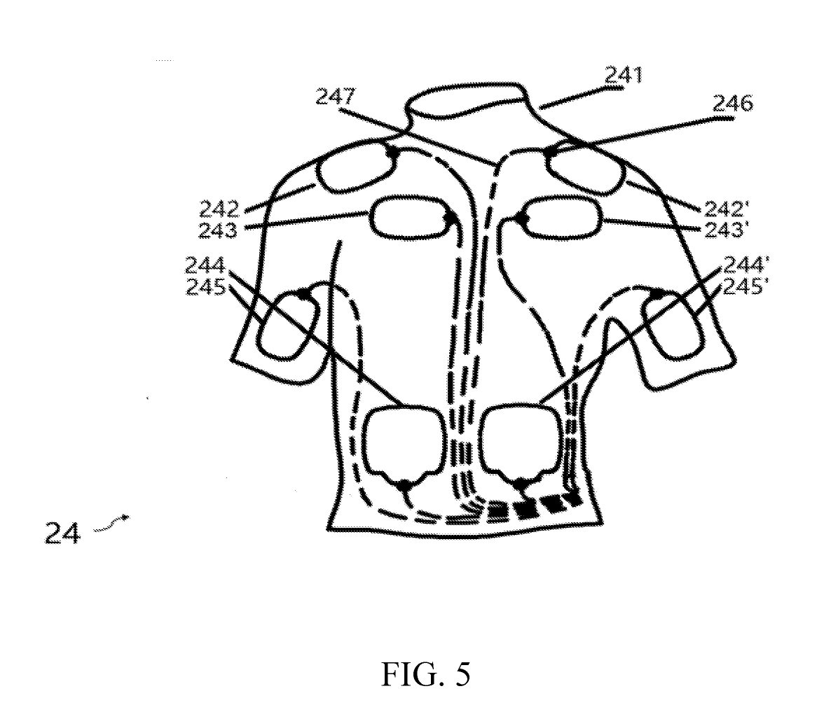

As shown in FIG. 5, in further another embodiment, the conductive shirt (24) comprises a main body (241) which is made of insulated fabric, and the main body (241) has multiple pairs of electrodes (242 and 242′, 243 and 243′, 244 and 244′, and 245 and 245′) which are made of conductive fabric and respectively located at different positions corresponding to user's upper body. Wherein each electrode secured or attached respectively on the main body (241) has a conductive engaging portion (246) which is connected thereon to a conductive wire (247). Through the conductive wires insulated between, each pair of electrodes is merged to connect to the corresponding pair of connectors on the control panel (1).

More specifically, since different stimulating accessories (2) are designed for corresponding portions of user's body, the distance between conductive fasteners may be different in different embodiments. Thus, in one embodiment, the surface of the control panel (1) has a plurality of conductive fasteners which are configured to engage with the conductive fasteners separated with the same or different distances on the electrodes. For example, the electrodes in the mentioned four embodiments are separated with different distances, such that the control panel (1) is configured to have four pairs of conductive fasteners separated with preferred distances to fit and engage with the conductive fasteners in different embodiments. In another embodiment, the control panel (1) is configured to have one or more pairs of conductive fasteners separated with preferred distances to allow each pair of conductive fasteners in different embodiments to be applied.

In one embodiment, the control panel (1) and stimulating accessory (2) are electrically connected through wires. Wherein the control panel (1) has electric connectors for connecting wires.

Comparing with conventional electrical stimulation apparatus, the present invention is advantageous because: (i) the electrical stimulation apparatus of the present invention is configured to be operated through a connected control terminal or directly through the control panel thereof; (ii) once the electrical stimulation apparatus is connected to a control terminal, the parameters in both the apparatus and control terminal are configured to be synchronized to facilitate use and adjustment; and (iii) the stimulating accessory is wearable, comfortable and convenient to use, compared to the conventional sticky gel electrode accessory that requires frequent replacements and spending.

Having described the invention by the description and illustrations above, it should be understood that these are exemplary of the invention and are not to be considered as limiting. Accordingly, the invention is not to be considered as limited by the foregoing description, but includes any equivalents.

What is claimed is:

1. An electrical stimulation apparatus comprising a control panel and a stimulating accessory;
wherein the control panel has a power supply, a control unit and a wireless communication unit, and a shell covers the control panel; wherein the control unit is configured to control the stimulating accessory, and the wireless communication unit is electrically connected to the control unit; a power switch, an interaction portion and connectors are respectively installed with the shell; wherein the power switch is configured to control the on/off operation of the power supply, and the interaction portion electrically connected to the control unit is configured to respond to operating signals sending from a user, and the connectors are configured to electrically connect to the stimulating accessory.

2. The electrical stimulation apparatus of claim 1, wherein the control unit comprises an IC chip which is preset to respond to operating signals from the interaction portion or a control terminal to control the stimulating accessory or to send parameters according to received signals back to the connected control terminal; and wherein the control terminal is connected to the electrical stimulation apparatus.

3. The electrical stimulation apparatus of claim 2, wherein the wireless communication unit comprises a Bluetooth, Wi-Fi, or radio frequency module and is configured to have bi-directional communication with the control terminal, and the wireless module is electrically connected with the IC chip.

4. The electrical stimulation apparatus of claim 3, wherein the control panel optionally comprises a display/indicator which is electrically connected to the control unit and configured to display control parameters of the control unit.

5. The electrical stimulation apparatus of claim 4, wherein the optional display/indicator comprises at least a LED light which is configured to be activated corresponding to control parameters of the control unit.

6. The electrical stimulation apparatus of claim 1, wherein the stimulating accessory has a main portion which is configured to be attached or covers on a whole or a portion of a user's body, and the main portion of the accessory has at least two electrodes which are insulated from each other; and wherein each of the electrodes has a connecting portion which is configured to electrically connect to the connector of the control panel.

7. The electrical stimulation apparatus of claim 6, wherein the stimulating accessory comprises two or more electrodes which are made of conductive soft materials, and each of the electrodes has a conductive engaging portion formed at an outer side thereof, and a conductive engaging slot formed at the connector of the control panel is configured to engage with the engaging portion of the electrode.

8. The electrical stimulation apparatus of claim 7, wherein the engaging portion of the electrode comprises at least a conductive fastener and a stub which are respectively formed at an outer surface and an inner surface of the electrode, and the engaging slot at the connector of the control panel has at least a conductive fastener which is configured to engage with the conductive fastener of the electrode; and wherein the conductive fastener on the electrode is secured through the stub.

9. The electrical stimulation apparatus of claim 8, wherein the stimulating accessory is made of at least a conductive soft material and at least an insulated soft material, which are sewed, knitted or attached together.

10. The electrical stimulation apparatus of claim 9, wherein the stimulating accessory is a conductive item which is wearable or configured to attach to a user's body, including a conductive sock, a conductive kneecap, a conductive piece of shorts, a conductive shirt, a conductive suit, a conductive glove, a conductive facial mask, a conductive shoe, a conductive arm/leg sleeve and a conductive waistband.
